(19) United States
(12) Patent Application Publication
(10) Pub. No.: US 2018/0368737 A1
(43) Pub. Date: Dec. 27, 2018

(54) PERSONALIZED FITNESS TRACKING

(71) Applicant: KONINKLIJKE PHILIPS N.V., EINDHOVEN (NL)

(72) Inventors: Alberto Giovanni Bonomi, Eindhoven (NL); Jenny Margarito, Eindhoven (NL)

(21) Appl. No.: 16/063,499

(22) PCT Filed: Jan. 6, 2017
(86) PCT No.: PCT/EP2017/050264

§ 371 Date: Jun. 18, 2018

Related U.S. Application Data
(60) Provisional application No. 62/276,413, filed on Jan. 8, 2016.

(51) Int. Cl. A61B 5/11; A61B 5/00; A61B 5/024; A61B 5/0255; A61B 5/22

(52) U.S. Cl. CPC A61B 5/1118; A61B 5/7264; A61B 5/22; A61B 5/02438; A61B 5/0255; A61B 5/02405

(57) ABSTRACT

In an embodiment, an apparatus that estimates cardiorespiratory fitness of a subject using data provided by wearable sensors in a free-living environment based on a physical performance measure indicated by a ratio between a measure of mechanical work and a measure of a physiological response associated with the mechanical work.

### FIELD OF THE INVENTION

The present invention is generally related to fitness tracking.

### BACKGROUND OF THE INVENTION

Cardiorespiratory fitness is an important health indicator that improves during lifestyle intervention and rehabilitation programs. Tracking fitness and fitness changes over time is challenging and is typically implemented via periodic execution of testing protocols by a user. Cardiorespiratory fitness is typically determined by measuring VO₂max, which corresponds to the maximum amount of oxygen uptake during physical activity. VO₂max depends on the ventilator capacity of the lungs, on the strength of the heart pumping function, and by the size of the muscle mass active during physical activity. This suggests that several body characteristics of a user can influence these factors. Body composition indicating muscle mass, gender, age, and body weight have been found to be strong predictors of VO₂max. Changes in VO₂max can be monitored by periodically performing maximal or sub-maximal exercise tests. This process is quite burdensome and provides insights into someone's fitness level only sporadically. Automatic (free-living, such as daily activity where a protocol is not required) methods to estimate VO₂max of an individual have been developed. For instance, WO 2015036289 A1 describes, as an object of the invention, a system, a method, a processor and a processing method for estimating cardiovascular fitness of a person (user) which do not require the user to rigorously follow a predetermined protocol and/or to use a certain equipment like a treadmill or ergometer, but allows the user to reliably assess his cardiovascular fitness in everyday life situations. The system includes a heart rate monitor for acquiring a heart rate signal and an activity monitor for acquiring an activity signal indicative of physical activity of the person, a classifier for classifying the activity of the person based on the acquired activity signal, a selector for selecting one or more heart rate features obtained from the acquired heart rate signal based on the acquired heart rate signal and the classification of the activity for use in an estimation of the cardiovascular fitness of the person, and an estimator for estimating the cardiovascular fitness of the person based on the one or more selected heart rate features. However, accuracy in fitness assessment depends heavily on the detail of the classification (see, e.g., page 4, lines 20-25), including running, raising, steady-state, recovery, cycling, etc. (see, e.g., page 14, lines 19-24), with further need for the heart rate feature to select (e.g., rising heart rate, stead-state exercise heart rate, heart rate recovery, etc., as described on page 11, lines 5-10) to predict VO₂ max. This need for contextual information appears to require a sophisticated activity classification system, which may be inaccurate, leading to inaccuracies of VO₂max estimations.

### SUMMARY OF THE INVENTION

One object of the present invention is to develop a fitness index that accurately reflects a measure of cardiovascular fitness (e.g., VO₂max) without a need for qualified personnel to monitor a rigorous fitness protocol, such as previously implemented in open-circuit spirometry during a maximal exercise test. Another object is to use a physical performance measure with a relationship to VO₂max that is valid across a wide range of activity types. To better address such concerns, in a first aspect of the invention, an apparatus is presented that estimates cardiorespiratory fitness using data provided by wearable sensors and based on a physical performance measure indicated by a ratio between a measure of mechanical work corresponding to free-living activity and a measure of a physiological response associated with the mechanical work. The invention addresses a problem in the art of a subject needing to adhere to stringent testing protocols and associated equipment, while also providing a performance measure that can be accurately applied in free-living activities of all types and not restricted to, for instance, running only, since the performance measure is largely activity-independent.

In one embodiment, a method is presented that estimates a change in the cardiorespiratory fitness measure for the subject based on changes in the physical performance and changes in an activity pattern and the estimated cardiorespiratory fitness measure. By using changes in physical performance, activity pattern, and the estimated cardiorespiratory fitness measure, a method is established for accurately and reliably assessing changes in fitness over time automatically without the need for further rigid testing and/or protocols.

In one embodiment, the data comprises movement data and physiological data corresponding to the subject, wherein determining physical performance comprises: determining activity type and motion intensity; determining heart rate based on the physiological data; and determining activity-dependent energy expenditure estimates based on the activity type and the motion intensity, wherein the physical performance comprises a ratio of the energy expenditure estimates and the heart rate. Determining VO₂ max from the ratio between energy expenditure and heart rate enables an assessment of fitness level from wearable sensor data during everyday (free-living) activities, including running, walking, cycling. In other words, the activity pattern is not restricted to a protocol or limited to a single activity. Further, the activity-independent nature of energy expenditure in determining activity intensity facilitates the free-living application, in that body movement or other easily-acquired measurements of intensity, including those achieved through the use of a pressure sensor, sweat sensor, etc., may be used. The energy expenditure need not be limited to heart rate, as multiple physiological parameters including respiration rate, skin temperature, galvanic skin response, etc., may be used to improve the veracity of the ratio.

In one embodiment, an activity pattern is determined from the free-living activities by categorizing the activity as sleep, sedentary, or active, the categorization based on the movement data and the physiological data. That is, using measurements from a wearable sensor, the activity pattern is monitored with little to no effort from the subject, while deriving accurate information about the nature of the subject activity. The activity pattern may be considered a summary metric for the activities carried out during a particular repeat period, for instance, each day, where not only walking activities are processed but also sedentary, anaerobic, and aerobic periods are assessed in order quantify the activity pattern of a subject.

In one embodiment, further comprising determining whether the active categorization corresponds to anaerobic or aerobic activity based on a duration of the activity and plural thresholds for the physiological data. Further delineating the activity as anaerobic or aerobic is helpful, as aerobic activities, when used for defining the activity pattern, correlate well with the maintenance and modification of the fitness level of the subject.

In one embodiment, wherein estimating the cardiorespiratory fitness measure comprises: estimating the cardiorespiratory fitness measure over the defined period of time for multiple types of activities for which the data is received and weighting each of the multiple types of activities according to a reliability score, the reliability score comprising correlation coefficients between the physical performance and the cardiorespiratory fitness measure. The use of multiple types of activities for the estimation of the cardiorespiratory fitness measure further highlights the value on the method in not being restricted to a protocol, but rather, basing the measure on free-living activities. Further, activities are not equally descriptive of VO₂max, and the use of reliability weights enables varying relevance to the predictions obtained during certain activities to account for these differences.

In one embodiment, further comprising estimating the cardiorespiratory fitness measure for plural successive defined periods of time for a duration of a baseline period. Providing daily estimates over a baseline period enables a determination of a reference starting level for weekly VO₂max estimates, and also enables the capture of variations in a subject's activity patterns influenced by week day and weekend day activities to provide more accurate and reliable assessments.

In one embodiment, further comprising estimating a change in the determined cardiorespiratory fitness measure for the subject based on changes in the physical performance and changes in an activity pattern and the estimated cardiorespiratory fitness measure after the baseline period. For instance, if the subject begins a training program after the baseline period, his or her aerobic activity increases, and the method determines the expected change over time in fitness according to the magnitude of the pattern change. One benefit to the estimate in change according to the changes in the physical performance and changes in an activity pattern and the estimated cardiorespiratory fitness measure is that an estimate of the change in the determined cardiorespiratory fitness measure is independent of the subject's characteristics. Accordingly, the determinant role of body characteristics in estimation of absolute VO₂max is overcome in the context of temporal changes to fitness levels.

In one embodiment, further comprising measuring an aerobic exercise response related to a measured pattern change. By measuring the aerobic exercise response, the method may be personalized to the subject's activities and behavioral response.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example environment in which a fitness tracking system is used in accordance with an embodiment of the invention.

FIG. 2 is a block diagram that illustrates circuitry for an example wearable device in accordance with an embodiment of the invention.

FIG. 3 is a block diagram that illustrates a processing circuit for an example computing device in accordance with an embodiment of the invention.

FIG. 4 is a flow diagram that illustrates an example method to determine cardiorespiratory fitness and changes in fitness from measurements of physical performance and activity patterns over time in accordance with an embodiment of the invention.

FIG. 5 is a flow diagram that illustrates an example method that is used to determine physical performance from measurements of body movement and physiological data such as heart rate in accordance with an embodiment of the invention.

FIG. 6 is a flow diagram that illustrates an example method that is used to determine activity and aerobic activity patterns from measurements of body movement and heart rate in accordance with an embodiment of the invention.

FIG. 7 are chart diagrams that illustrate that for certain activity types, like running, a stronger relationship exists between physical performance (TEE/HR) and fitness (VO₂max).

FIGS. 8-9 are chart diagrams that respectively illustrate an example effect of training time on percentage change in VO₂max according to the type of exercise and an example effect of training cessation on percentage change in VO₂max.

FIG. 10 is a schematic diagram that illustrates an example expected trend in fitness over time as determined by a fitness training program inducing a specific pattern change in user routine in accordance with an embodiment of the invention.

FIG. 11 is a schematic diagram that conceptually illustrates a method to improve reliability of daily estimates of VO₂max based on the expected fitness trend over time as determined by changes in activity patterns, for example due to intervention programs, in accordance with an embodiment of the invention.

FIG. 12 is a schematic diagram that conceptually illustrates a method used to personalize parameters of a model used to estimate temporal trends in fitness as determined by the change in patterns in accordance with an embodiment of the invention.

### DETAILED DESCRIPTION OF EMBODIMENTS

Disclosed herein are certain embodiments of a fitness tracking system, apparatus, and method (collectively hereinafter referred to as a fitness tracking system) that provide for accurate and reliable estimates of cardiorespiratory fitness and changes thereto. In one embodiment, a fitness tracking method is disclosed that is protocol-free and that estimates the cardiorespiratory fitness of a subject based on monitored physiological data (e.g., heart rate and body movement data) under free-living conditions, for instance through implementation of a fitness index that reflects a measure of cardiorespiratory fitness (e.g., VO₂max) versus heart rate relationship of the subject by combining acceleration-based estimates of energy expenditure with heart rate data. In some embodiments, a fitness tracking method further defines baseline characteristics of a user including physical performance and types of activities and activity patterns, provides context-dependent assessment of an initial VO₂max according to the baseline characteristics, estimates changes in VO₂max according to recorded changes in activity patterns, physical performance, and the initial VO₂max, and estimates an aerobic exercise response to further personalize models predicting changes in VO₂max over time from activity patterns. When aiming at determining changes in cardiorespiratory fitness, subjects' characteristics are irrelevant predictors, as they remain relatively stable over time. In certain embodiments of fitness tracking systems, physical performance and activity or exercise patterns are relevant features to describe changes in VO₂max over time. Physical performance may change in agreement with variation in VO₂max. Likewise, exercise routine and activity patterns may induce specific changes in cardiorespiratory fitness. Certain embodiments of a fitness tracking system determine cardiorespiratory fitness and changes in fitness over time by using data collected with wearable sensors.

Note that the determination of cardiorespiratory fitness measures (and changes in cardiorespiratory fitness measures) are based on data gathered for a subject engaged in a free-living, activity pattern over a defined period of time. Free-living may in some embodiments involve daily activity where the subject is not encumbered by rigorous protocols, such as in a clinical or laboratory setting. Nevertheless, the subject may be involved in a training regime that is supervised (e.g., by a trainer or coach) or unsupervised, with the intent that such activity still rises to the level of a free-living activity pattern, since it is not subject to constraints in a laboratory or clinical setting.

Certain embodiments of fitness tracking systems use a fitness index that is defined by combining acceleration and heart rate data. For instance, a total energy expenditure (TEE)-pulse parameter is determined as a ratio between TEE and HR for a given time period (e.g., sixty (60) seconds, though other periods of time may be used). The fitness index (e.g., TEE/HR or TEE-pulse) is highly indicative for oxygen-pulse, which is an index of physical performance and correlates with VO₂max (particularly for each activity type as described below). Digressing briefly, because of the difficulties associated with maximal exercise testing, many submaximal tests have been developed to estimate cardiorespiratory fitness. For instance, estimation of VO₂max from submaximal tests is based on a linear relationship between oxygen uptake (VO₂) and mechanical power output, or heart rate. Tests require the participants to undergo an activity protocol and may require specific exercise equipment. Some submaximal tests are suitable for self-evaluation, yet the accuracy and reproducibility of the estimates provided by such methods are lower than those offered by direct measures of VO₂max. In contrast, the fitness index (TEE/HR) used to estimate cardiorespiratory fitness in the disclosed embodiments of fitness tracking systems is based on wearable sensor data, highly correlated to VO₂max, and does not require a specific exercise protocol. As is described below, embodiments of a fitness tracking system identify a contextual situation during which measurements of energy expenditure and heart rate are obtained in free-living conditions to generate a fitness index and predict VO₂max (and changes in VO₂max). By combining the fitness index with contextual information on activity type and intensity as derived from wearable sensor data, the development of a highly accurate VO₂max prediction mechanism is achieved.

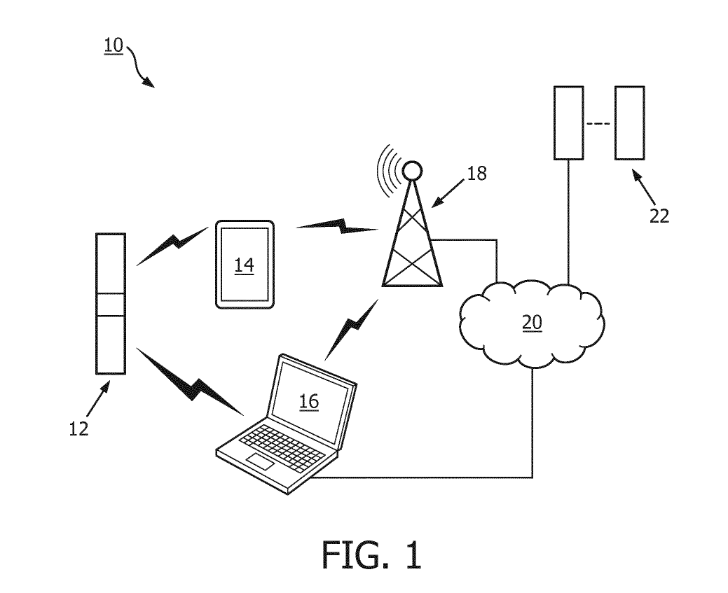

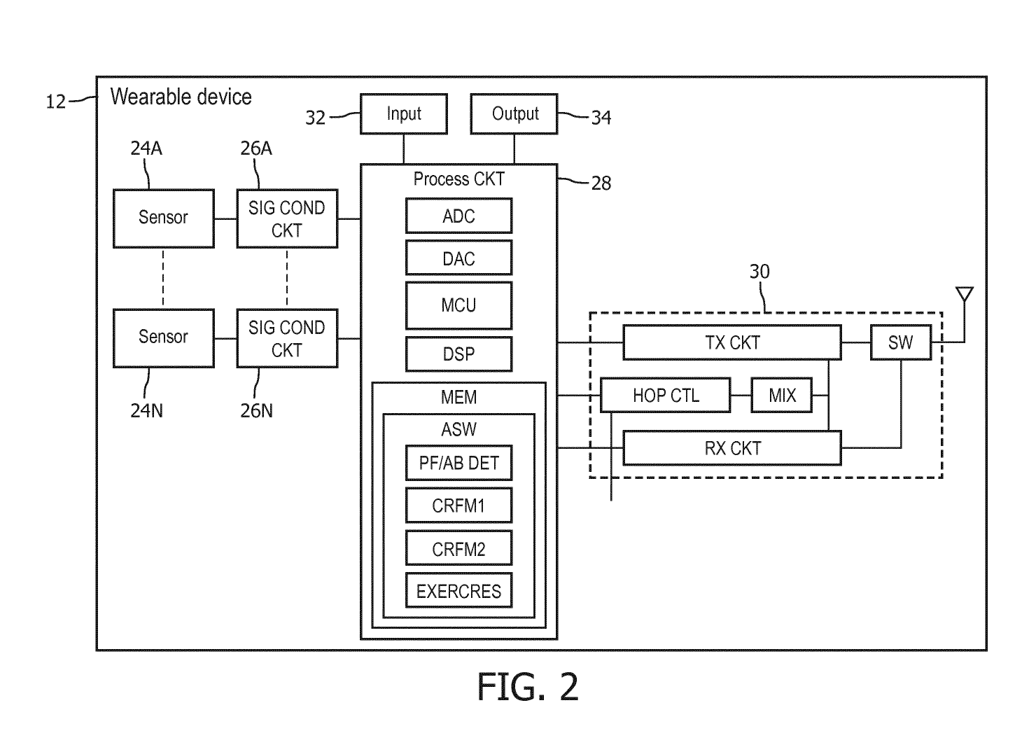

Attention is directed to FIG. 1, which illustrates an example environment 10 in which a fitness tracking system is used in accordance with an embodiment of the invention. It should be appreciated by one having ordinary skill in the art in the context of the present disclosure that the environment 10 is one example among many, and that some embodiments of a fitness tracking system may be used in environments with fewer, greater, and/or different components than those depicted in FIG. 1. The environment 10 comprises a plurality of devices that enable communication of information throughout one or more networks. The depicted environment 10 comprises a wearable device 12, electronics devices 14, 16, a cellular network 18, a wide area network 20 (e.g., also described herein as the Internet), and a remote computing system 22. The wearable device 12, as described further in association with FIG. 2, is typically worn by the subject (e.g., around the wrist, arm, torso, etc.), and comprises a plurality of sensors (wearable sensors) that track physical activity of the subject (e.g., steps, swim strokes, pedaling strokes, etc.), sense or derive physiological parameters (e.g., heart rate, respiration, skin temperature, etc.) based on the sensor data, and optionally sense various other parameters (e.g., outdoor temperature, humidity, location, etc.) pertaining to the surrounding environment of the wearable device 12. A representation of such gathered data may be communicated to the subject via an integrated display on the wearable device and/or on another device or devices.

Also, such data gathered by the wearable device 12 may be communicated (e.g., continually, periodically, and/or aperiodically) to one or more electronics devices, including the electronics devices 14 and 16. Such communication may be achieved wirelessly (e.g., using near field communications (NFC) functionality, Bluetooth functionality, etc.) and/or according to a wired medium (e.g., universal serial bus (USB), etc.). In the depicted example, the electronics device 14 is embodied as a phone and the electronics device 16 is embodied as a computer. It should be appreciated that although each electronics device is listed in the singular, some implementations may utilize different quantities for each of the electronics devices 14, 16. Further, in some embodiments, fewer, additional, and/or other types of electronics devices may be used. The phone 14 may be embodied as a smartphone, mobile phone, cellular phone, pager, among other handheld computing/communication devices with telephony or communication functionality. For the sake of example, assume the phone 14 is embodied as a smartphone. The smartphone 14 comprises at least two different processors, including a baseband processor and an application processor. The baseband processor comprises a dedicated processor for deploying functionality associated with a protocol stack, such as a GSM (Global System for Mobile communications) protocol stack. The application processor comprises a multi-core processor for providing a user interface and running applications. The baseband processor and application processor have respective associated memory (e.g., random access memory (RAM), Flash memory, etc.), peripherals, and a running clock.

More particularly, the baseband processor may deploy functionality of a GSM protocol stack to enable the smartphone 14 to access one or a plurality of wireless network technologies, including WCDMA (Wideband Code Division Multiple Access), CDMA (Code Division Multiple Access), EDGE (Enhanced Data Rates for GSM Evolution), GPRS (General Packet Radio Service), Zigbee (e.g., based on IEEE 802.15.4), Bluetooth, Wi-Fi (Wireless Fidelity, such as based on IEEE 802.11), and/or LTE (Long Term Evolution), among variations thereof and/or other telecommunication protocols, standards, and/or specifications. The baseband processor manages radio communications and control functions, including signal modulation, radio frequency shifting, and encoding. The baseband processor may comprise a GSM modem having one or more antennas, a radio (e.g., RF front end), and analog and digital baseband circuitry. The RF front end comprises a transceiver and a power amplifier to enable the receiving and transmitting of signals of a plurality of different frequencies, enabling access to the cellular network 18. The analog baseband is coupled to the radio and provides an interface between the analog and digital domains of the GSM modem. The analog baseband comprises circuitry including an analog-to-digital converter (ADC) and digital-to-analog converter (DAC), as well as control and power management/distribution components and an audio codec to process analog and/or digital signals received from the smartphone user interface (e.g., microphone, earpiece, ring tone, vibrator circuits, etc.). The ADC digitizes any analog signals for processing by the digital baseband processor. The digital baseband processor deploys the functionality of one or more levels of the GSM protocol stack (e.g., Layer 1, Layer 2, etc.), and comprises a microcontroller (e.g., microcontroller unit or MCU) and a digital signal processor (DSP) that communicate over a shared memory interface (the memory comprising data and control information and parameters that instruct the actions to be taken on the data processed by the application processor). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband and perform detection, demodulation, and decoding with reporting back to the MCU. For transmit-side functionality, the MCU presents transmittable data and auxiliary information to the DSP, which encodes the data and provides to the analog baseband (e.g., converted to analog signals by the DAC). The application processor may be embodied as a System on a Chip (SOC), and supports a plurality of multimedia related features including web browsing to access one or more computing devices of the computing system 22 that are coupled to the Internet, email, multimedia entertainment, games, etc.

The application processor includes an operating system that enables the implementation of a plurality of user applications. For instance, the application processor may deploy interface software (e.g., middleware, such as a browser with or operable in association with one or more application program interfaces (APIs)) to enable access to a cloud computing framework or other networks to provide remote data access/storage/processing, and through cooperation with an embedded operating system, access to calendars, location services, reminders, etc. For instance, in some embodiments, a fitness tracking system may operate using cloud computing, where the processing and storage of user data and the determination of physical performance, activity patterns, VO₂max, changes in VO₂max, etc. may be achieved by one or more devices of the computing system 22. The application processor generally comprises a processor core (Advanced RISC Machine or ARM), multimedia modules (for decoding/encoding pictures, video, and/or audio), a graphics processing unit (GPU), wireless interfaces, and device interfaces. The wireless interfaces may include a Bluetooth or Zigbee module(s) that enables wireless communication with the wearable device 12 or other local devices, a Wi-Fi module for interfacing with a local 802.11 network, and a GSM module for access to the cellular network 18 and via browser functionality the wide area network 20. The device interfaces coupled to the application processor may include a respective interface for such devices as a display screen. The display screen may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), or retina or haptic-based technology. For instance, the display screen may be used to present web pages and/or other documents received from the computing system 22 and/or in some embodiments (e.g., for local processing) graphic user interfaces (GUIs) rendered locally, either of which may present feedback in the form of a visual representation of a physical performance and/or fitness levels and associated data. Other interfaces include a keypad, USB (Universal Serial Bus), SD/MMC card, camera, GPRS, Wi-Fi, GPS, and/or FM radios, memory, among other devices. It should be appreciated by one having ordinary skill in the art, in the context of the present disclosure, that variations to the above may be deployed in some embodiments to achieve similar functionality.

The computer 16 may be embodied as a laptop, personal computer, workstation, personal digital assistant, tablet, among other computing devices with communication capability. The computer 16 may be in wireless or wired (e.g., temporarily, such as via USB connection, or persistently, such as an Internet connection or local area network connection) communication with other devices. The computer 16 may include similar hardware and software/firmware to that described above for the phone 14 to enable access to wireless and/or cellular networks (e.g., through communication cards comprising radio and/or cellular modem functionality) and/or other devices (e.g., Bluetooth transceivers, NFC transceivers, etc.), such as wireless or (temporary) wired connection to the wearable device 12. In some implementations, the computer 16 may be coupled to the Internet 20 through the plain old telephone service (POTS), using technologies such as digital subscriber line (DSL), asymmetric DSL (ADSL), and/or according to broadband technology that uses a coaxial, twisted pair, and/or fiber optic medium. Discussion of such communication functionality is omitted here for brevity. Generally, in terms of hardware architecture, the computer 16 includes a processor, memory, and one or more input and/or output (I/O) devices (or peripherals) that are communicatively coupled via a local interface. The local interface can be, for example but not limited to, one or more buses or other wired or wireless connections. The local interface may have additional elements, which are omitted for brevity, such as controllers, buffers (caches), drivers, repeaters, and receivers, to enable communications. Further, the local interface may include address, control, and/or data connections to enable appropriate communications among the aforementioned components.

The processor is a hardware device for executing software, particularly that stored in memory. The processor can be any custom made or commercially available processor, a central processing unit (CPU), an auxiliary processor among several processors associated with the computer 16, a semiconductor based microprocessor (in the form of a microchip or chip set), a macroprocessor, or generally any device for executing software instructions.

The memory can include any one or combination of volatile memory elements (e.g., random access memory (RAM, such as DRAM, SRAM, SDRAM, etc.) and nonvolatile memory elements (e.g., ROM, hard drive, Flash, EPROM, EEPROM, CDROM, etc.). Moreover, the memory may incorporate electronic, magnetic, optical, semi-conductive, and/or other types of storage media. Note that the memory can have a distributed architecture, where various components are situated remote from one another, but can be accessed by the processor.

The software in memory may include one or more separate programs, such as interface software (e.g., middleware, such as browser software with or associated with one or more APIs) to communicate with other network devices, such as one or more devices of the computing system 22, the separate programs each comprising an ordered listing of executable instructions for implementing logical functions. The software in the memory also includes application software and a suitable operating system (O/S). The operating system may be embodied as a Windows operating system available from Microsoft Corporation, a Macintosh operating system available from Apple Computer, a UNIX operating system, among others. The operating system essentially controls the execution of other computer programs, and provides scheduling, input-output control, file and data management, memory management, and communication control and related services.

The I/O devices may include input devices, for example but not limited to, a keyboard, mouse, scanner, microphone, etc. Furthermore, the I/O devices may also include output devices, for example but not limited to, a printer, display, etc. For instance, the I/O devices embodied as a display screen may be used to present web pages and/or other documents received from the computing system 22 and/or in some embodiments (e.g., for local processing) graphic user interfaces (GUIs) rendered locally, either of which may present feedback in the form of a visual representation of the physical performance, activity patterns, VO₂max, changes in VO₂max, etc. The display screen may be configured according to any one of a variety of technologies, including cathode ray tube (CRT), liquid crystal display (LCD), plasma, haptic, among others well-known to those having ordinary skill in the art.

If the computer is a PC, workstation, or the like, the software in the memory may further include a basic input output system (BIOS). The BIOS is a set of essential software routines that initialize and test hardware at startup, start the O/S, and support the transfer of data among the hardware devices. The BIOS is stored in ROM so that the BIOS can be executed when the computer 16 is activated.

When the computer 16 is in operation, the processor is configured to execute the software stored within the memory, to communicate data to and from the memory, and to generally control operations of the computer 16 pursuant to the software. Software can be stored on any non-transitory computer readable medium for use by or in connection with any computer related system or method. In the context of this document, a computer readable medium comprises an electronic, magnetic, optical, electromagnetic, infrared, or semiconductor system, apparatus, device or means that can contain or store a computer program for use by or in connection with a computer related system or method. The software can be embodied in any non-transitory computer-readable medium for use by or in connection with an instruction execution system, apparatus, or device, such as a computer-based system, processor-containing system, or other system that can fetch the instructions from the instruction execution system, apparatus, or device and execute the instructions.

The cellular network 18 may include the necessary infrastructure to enable cellular communications by the phone 14 and optionally the computer 16. There are a number of different digital cellular technologies suitable for use in the cellular network 18, including: GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others.

The wide area network 20 may comprise one or a plurality of networks that in whole or in part comprise the Internet. The electronics devices 14, 16 access the devices of the computing system 22 via the Internet 20, which may be further enabled through access to one or more networks including PSTN (Public Switched Telephone Networks), POTS, Integrated Services Digital Network (ISDN), Ethernet, Fiber, DSL/ADSL, among others.

The computing system 22 comprises a plurality of devices coupled to the wide area network 20, including one or more computing devices such as application servers, a computer network, and data storage. As described previously, the computing system 22 may serve as a cloud computing environment (or other server network) for the electronics devices 14, 16, performing processing and data storage on behalf of (or in some embodiments, in addition to) the electronics devices 14, 16 and/or the wearable device 12. In some embodiments, one or more of the functionality of the computing system 22 may be performed at the respective electronics devices 14, 16, and/or wearable device 12 and vice versa.

An embodiment of a fitness tracking system may comprise the wearable device 12, or in some embodiments, a combination of the wearable device 12 and one or more other devices (or equivalently, one or more apparatuses) depicted in the environment 10 (e.g., the electronics devices 14, 16, and/or devices of the computing system 22). In some embodiments, the fitness tracking system may be implemented on one of the other devices, such as one of the electronics devices 14,16 or a device or devices of the computing system 22. In the description that follows, a focus is on an implementation where functionality of the fitness tracking system is implemented in the wearable device 12, with the understanding that the functionality may be implemented in one or more other devices and/or additional devices of the environment 10.

Having generally described an example environment 10 in which an embodiment of a fitness tracking system may be implemented, attention is directed to FIG. 2. FIG. 2 illustrates example circuitry for the example wearable device 12, and in particular, underlying circuitry and software (e.g., architecture) of the wearable device 12 that in one embodiment is used to implement a fitness tracking system. It should be appreciated by one having ordinary skill in the art in the context of the present disclosure that the architecture of the wearable device 12 depicted in FIG. 2 is but one example, and that in some embodiments, additional, fewer, and/or different components may be used to achieve similar and/or additional functionality. In one embodiment, the wearable device 12 comprises a plurality of sensors 24 (e.g., 24A-24N, also referred to as wearable sensors), one or more signal conditioning circuits 26 (e.g., SIG COND CKT 26A—SIG COND CKT 26N) coupled respectively to the sensors 24, and a processing circuit 28 (PROCES CKT) that receives the conditioned signals from the signal conditioning circuits 26. In one embodiment, the processing circuit 28 comprises an analog-to-digital converter (ADC), a digital-to-analog converter (DAC), a microcontroller (e.g., MCU), a digital signal processor (DSP), and memory (MEM), including the software in memory. In some embodiments, the processing circuit 28 may comprise fewer or additional components than those depicted in FIG. 2. For instance, in one embodiment, the processing circuit 28 may consist of the microcontroller. The memory comprises an operating system (OS) and application software (ASW). The application software comprises a plurality of algorithms (e.g., application modules of executable code) to process the signals (and associated data) measured by the sensors and record and/or derive physiological parameters, such as heart rate, blood pressure, respiration, perspiration, etc. The application software (ASW) further comprises one or more modules of a fitness tracking system to determine physical performance and activity patterns of a subject (e.g., user or animal) that possesses the wearable device 12 (PF/AB DET in FIG. 2), provide a context-dependent estimate of a cardiorespiratory fitness measure (e.g., an initial VO₂max) that is estimated based on the physical performance, activity categories, and characteristics of the user (e.g., gender, age, weight, body mass index, height, etc.) (CRFM1), provide an estimate of changes in the estimated initial VO₂max based on recorded changes in activity patterns, physical performance, and the initial VO₂max (collectively performed by CRFM2), and estimate an aerobic exercise response that personalizes models used to predict changes in VO₂max over time from the activity patterns (EXERCRES). As described further below, an equation, αVO₂max (e.g., computed by CRFM2, of which EXERCRES is associated) is used to generate personalized parameters to input to the model predicting changes in VO₂max according to changes in aerobic activity patterns. A differential equation of the VO₂max temporal prediction model evaluated during a first period (e.g., first week) of intervention determines the subject-specific period, T, which indicates the time period (e.g., in weeks) needed to reach about 63% of the asymptotic (expected) final VO₂max as a result of change in aerobic patterns. Without the functionality associated with EXERCRES, the T parameter is taken from literature on population statistics defining an average amount of time needed to reach 63% of the final VO₂max. The models linking the changes in aerobic activity patterns to changes in VO₂max are initially based on population statistics and subsequently personalized according to daily estimates. In other words, the output of CRFM2 is modified by the outcome of EXERCRES.

The application software also comprises communications software, such as that used to enable the wearable device 12 to operate according to one or more of a plurality of different communication technologies (e.g., NFC, Bluetooth, Wi-Fi, Zigbee, etc.). In some embodiments, the communications software may be in separate or other memory.

The memory further comprises one or more data structures. In one embodiment, the processing circuit 28 is coupled to a communications circuit 30. The communications circuit 30 serves to enable wireless communications between the wearable device 12 and other electronics devices, such as the phone 14, the laptop 16, and/or other devices. The communications circuit 30 is depicted as a Bluetooth circuit, though not limited to this transceiver configuration. For instance, in some embodiments, the communications circuit 30 may be embodied as any one or a combination of an NFC circuit, Wi-Fi circuit, transceiver circuitry based on Zigbee, among others such as optical or ultrasonic based technologies. The processing circuit 28 is further coupled to input/output (I/O) devices or peripherals, such as an input interface 32 (INPUT) and output interface 34 (OUT). Note that in some embodiments, functionality for one or more of the aforementioned circuits and/or software may be combined into fewer components/modules, or in some embodiments, further distributed among additional components/modules. For instance, the processing circuit 28 may be packaged as an integrated circuit that includes the microcontroller, the DSP, and memory, whereas the ADC and DAC may be packaged as a separate integrated circuit coupled to the processing circuit 28. In some embodiments, one or more of the functionality for the above-listed components may be combined, such as functionality of the DSP performed by the microcontroller.

The sensors 24 (hereinafter, also referred to as wearable sensors) are selected to perform detection and measurement of a plurality of physiological and behavioural or pattern parameters, including heart rate, heart rate variability, heart rate recovery, blood flow rate, activity level, muscle activity (e.g., movement of limbs, repetitive movement, core movement, body orientation/position, power, speed, acceleration, etc.), muscle tension, blood volume, blood pressure, blood oxygen saturation, respiratory rate, perspiration, skin temperature, body weight, and body composition (e.g., body mass index or BMI). The sensors 24 may be embodied as inertial sensors (e.g., gyroscopes, single or multi-axis accelerometers, such as those using piezoelectric, piezoresistive or capacitive technology in a microelectromechanical system (MEMS) infrastructure), flex and/or force sensors (e.g., using variable resistance), electromyographic sensors, electrocardiographic sensors (e.g., EKG, ECG) magnetic sensors, photoplethysmographic (PPG) sensors, bio-impedance sensors, infrared proximity sensors, acoustic/ultrasonic/audio sensors, a strain gauge, galvanic skin/sweat sensors, pH sensors, temperature sensors, pressure sensors, and photocells. In some embodiments, other types of sensors 24 may be used to facilitate health and/or fitness related computations, including a global navigation satellite systems (GNSS) sensor (e.g., global positioning system (GPS) receiver) to facilitate determinations of distance, speed, acceleration, location, altitude, etc. (e.g., location data and movement), barometric pressure, humidity, outdoor temperature, etc. In some embodiments, GNSS functionality may be achieved via the communications circuit 30 or other circuits coupled to the processing circuit 28.

The signal conditioning circuits 26 include amplifiers and filters, among other signal conditioning components, to condition the sensed signals including data corresponding to the sensed physiological parameters before further processing is implemented at the processing circuit 28. Though depicted in FIG. 2 as respectively associated with each sensor 24, in some embodiments, fewer signal conditioning circuits 26 may be used (e.g., shared for more than one sensor 24). In some embodiments, the signal conditioning circuits 26 (or functionality thereof) may be incorporated elsewhere, such as in the circuitry of the respective sensors 24 or in the processing circuit 28 (or in components residing therein). Further, although described above as involving unidirectional signal flow (e.g., from the sensor 24 to the signal conditioning circuit 26), in some embodiments, signal flow may be bi-directional. For instance, in the case of optical measurements, the microcontroller may cause an optical signal to be emitted from a light source (e.g., light emitting diode(s) or LED(s)) in or coupled to the circuitry of the sensor 24, with the sensor 24 (e.g., photocell) receiving the reflected/refracted signals.

The communications circuit 30 is managed and controlled by the processing circuit 28. The communications circuit 30 is used to wirelessly interface with the electronics devices 14, 16 (FIG. 1). In one embodiment, the communications circuit 30 may be configured as a Bluetooth transceiver, though in some embodiments, other and/or additional technologies may be used, such as Wi-Fi, Zigbee, NFC, among others. In the embodiment depicted in FIG. 2, the communications circuit 30 comprises a transmitter circuit (TX CKT), a switch (SW), an antenna, a receiver circuit (RX CKT), a mixing circuit (MIX), and a frequency hopping controller (HOP CTL). The transmitter circuit and the receiver circuit comprise components suitable for providing respective transmission and reception of an RF signal, including a modulator/demodulator, filters, and amplifiers. In some embodiments, demodulation/modulation and/or filtering may be performed in part or in whole by the DSP. The switch switches between receiving and transmitting modes. The mixing circuit may be embodied as a frequency synthesizer and frequency mixers, as controlled by the processing circuit 28. The frequency hopping controller controls the hopping frequency of a transmitted signal based on feedback from a modulator of the transmitter circuit. In some embodiments, functionality for the frequency hopping controller may be implemented by the microcontroller or DSP. Control for the communications circuit 30 may be implemented by the microcontroller, the DSP, or a combination of both. In some embodiments, the communications circuit 30 may have its own dedicated controller that is supervised and/or managed by the microcontroller.

In operation, a signal (e.g., at 2.4 GHz) may be received at the antenna and directed by the switch to the receiver circuit. The receiver circuit, in cooperation with the mixing circuit, converts the received signal into an intermediate frequency (IF) signal under frequency hopping control attributed by the frequency hopping controller and then to baseband for further processing by the ADC. On the transmitting side, the baseband signal (e.g., from the DAC of the processing circuit 28) is converted to an IF signal and then RF by the transmitter circuit operating in cooperation with the mixing circuit, with the RF signal passed through the switch and emitted from the antenna under frequency hopping control provided by the frequency hopping controller. The modulator and demodulator of the transmitter and receiver circuits may be frequency shift keying (FSK) type modulation/demodulation, though not limited to this type of modulation/demodulation, which enables the conversion between IF and baseband. In some embodiments, demodulation/modulation and/or filtering may be performed in part or in whole by the DSP. The memory stores firmware that is executed by the microcontroller to control the Bluetooth transmission/reception.

Though the communications circuit 30 is depicted as an IF-type transceiver, in some embodiments, a direct conversion architecture may be implemented. As noted above, the communications circuit 30 may be embodied according to other and/or additional transceiver technologies, such as NFC, Wi-Fi, or Zigbee.

The processing circuit 28 is depicted in FIG. 2 as including the ADC and DAC. For sensing functionality, the ADC converts the conditioned signal from the signal conditioning circuit 26 and digitizes the signal for further processing by the microcontroller and/or DSP. The ADC may also be used to convert analogs inputs that are received via the input interface 32 to a digital format for further processing by the microcontroller. The ADC may also be used in baseband processing of signals received via the communications circuit 30. The DAC converts digital information to analog information. Its role for sensing functionality may be to control the emission of signals, such as optical signals or acoustic signal, from the sensors 24. The DAC may further be used to cause the output of analog signals from the output interface 34. Also, the DAC may be used to convert the digital information and/or instructions from the microcontroller and/or DSP to analog signal that are fed to the transmitter circuit. In some embodiments, additional conversion circuits may be used.

The microcontroller and the DSP provide the processing functionality for the wearable device 12. In some embodiments, functionality of both processors may be combined into a single processor, or further distributed among additional processors. The DSP provides for specialized digital signal processing, and enables an offloading of processing load from the microcontroller. The DSP may be embodied in specialized integrated circuit(s) or as field programmable gate arrays (FPGAs). In one embodiment, the DSP comprises a pipelined architecture, which comprises a central processing unit (CPU), plural circular buffers and separate program and data memories according to a Harvard architecture. The DSP further comprises dual busses, enabling concurrent instruction and data fetches. The DSP may also comprise an instruction cache and I/O controller, such as those found in Analog Devices SHARC® DSPs, though other manufacturers of DSPs may be used (e.g., Freescale multi-core MSC81xx family, Texas Instruments C6000 series, etc.). The DSP is generally utilized for math manipulations using registers and math components that may include a multiplier, arithmetic logic unit (ALU, which performs addition, subtraction, absolute value, logical operations, conversion between fixed and floating point units, etc.), and a barrel shifter. The ability of the DSP to implement fast multiply-accumulates (MACs) enables efficient execution of Fast Fourier Transforms (FFTs) and Finite Impulse Response (FIR) filtering. The DSP generally serves an encoding and decoding function in the wearable device 12. For instance, encoding functionality may involve encoding commands or data corresponding to transfer of information to the electronics devices 14, 16. Also, decoding functionality may involve decoding the information received from the sensors 24 (e.g., after processing by the ADC).

The microcontroller comprises a hardware device for executing software/firmware, particularly that stored in memory. The microcontroller can be any custom made or commercially available processor, a central processing unit (CPU), a semiconductor based microprocessor (in the form of a microchip or chip set), a macroprocessor, or generally any device for executing software instructions. Examples of suitable commercially available microprocessors include Intel's® Itanium® and Atom® microprocessors, to name a few non-limiting examples. The microcontroller provides for management and control of the wearable device 12, including determining physiological parameters based on the sensors 24, and for enabling communication with the electronics devices 14, 16.

The memory can include any one or combination of volatile memory elements (e.g., random access memory (RAM, such as DRAM, SRAM, SDRAM, etc.)) and nonvolatile memory elements (e.g., ROM, Flash, solid state, EPROM, EEPROM, etc.). Moreover, the memory may incorporate electronic, magnetic, and/or other types of storage media.

The software in memory may include one or more separate programs, each of which comprises an ordered listing of executable instructions for implementing logical functions. In the example of FIG. 2, the software in the memory includes a suitable operating system and application software that includes a plurality of algorithms for determining physiological and/or behavioural or pattern measures and/or activity measures, physical performance, total energy expenditure (activity specific), among other information (e.g., such as location) based on the output from the sensors 24. The raw data from the sensors 24 may be used by the algorithms to determine various physiological and/or behavioural or pattern measures (e.g., heart rate, biomechanics, such as swinging of the arms), and may also be used to derive other parameters, such as energy expenditure, heart rate recovery, aerobic capacity (e.g., VO2 max, etc.), among other derived measures of physical performance. In some embodiments, these derived parameters may be computed externally (e.g., at the electronics devices 14, 16 or one or more devices of the computing system 22) in lieu of, or in addition to, the computations performed local to the wearable device 12. The application software may also include communications software to enable communications with other electronics devices. The operating system essentially controls the execution of other computer programs, such as the application software and communications software, and provides scheduling, input-output control, file and data management, memory management, and communication control and related services. The memory may also include a data structure, which includes user data (e.g., referred to herein also as user-specific information, or user characteristics), such as weight, height, age, gender, body mass index (BMI) that is used by the microcontroller executing the executable code of the algorithms to accurately interpret the measured physiological and/or behavioural or pattern data. In some embodiments, the data structure of user data may be stored elsewhere, such as at the electronics devices 14, 16 and/or at one or more devices of the computing system 22 in lieu of, or in addition to being stored at the wearable device 12.

The software in memory comprises a source program, executable program (object code), script, or any other entity comprising a set of instructions to be performed. When a source program, then the program may be translated via a compiler, assembler, interpreter, or the like, so as to operate properly in connection with the operating system. Furthermore, the software can be written as (a) an object oriented programming language, which has classes of data and methods, or (b) a procedure programming language, which has routines, subroutines, and/or functions, for example but not limited to, C, C++, Python, Java, among others. The software may be embodied in a computer program product, which may be a non-transitory computer readable medium or other medium.

The input interface 32 comprises an interface for entry of user input, such as a button or microphone or sensor (e.g., to detect user input). The input interface 32 may serve as a communications port for downloaded information to the wearable device 12 (such as via a wired connection). The output interfaces 34 comprises an interface for the presentation or transfer of data, such as a display screen, speaker, and/or communications interface for the transfer (e.g., wired) of information stored in the memory, or to enable one or more feedback devices, such as lighting devices (e.g., LEDs), audio devices (e.g., tone generator and speaker), and/or tactile feedback devices (e.g., vibratory motor). In some embodiments, at least some of the functionality of the input and output interfaces 32 and 34 may be combined, such as in the case of a touch-type display screen.

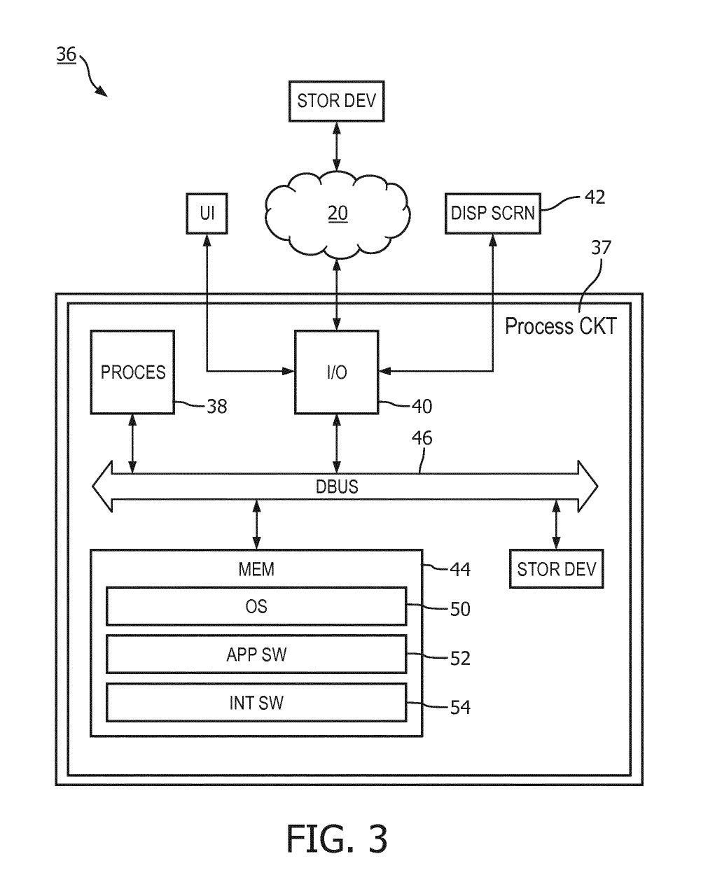

Having described the underlying hardware and software of the wearable device 12, attention is now directed to FIG. 3, which illustrates circuitry for an example computing device 36 of the computing system 22, in accordance with an embodiment of the invention. The computing device 36 may be embodied as an application server, computer, among other computing devices, and is also generally referred to herein as an apparatus. One having ordinary skill in the art should appreciate in the context of the present disclosure that the example computing device 36 is merely illustrative of one embodiment, and that some embodiments of computing devices may comprise fewer or additional components, and/or some of the functionality associated with the various components depicted in FIG. 3 may be combined, or further distributed among additional modules or computing devices, in some embodiments. The computing device 36 is depicted in this example as a computer system, such as one providing a function of an application server. It should be appreciated that certain well-known components of computer systems are omitted here to avoid obfuscating relevant features of the computing device 36. In one embodiment, the computing device 36 comprises a processing circuit 37 (PROCES CKT) that comprises one or more processors, such as processor 38 (PROCES), input/output (I/O) interface(s) 40 (I/O), which in one embodiment is optionally coupled to a display screen 42 (DISP SCRN) and other user interfaces (e.g., keyboard, mouse, microphone, etc.), and memory 44 (MEM), all coupled to one or more data busses, such as data bus 46 (DBUS). In some embodiments, the display screen 42 (and/or user interface (UI)) may be coupled directly to the data bus 46. The memory 44 may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.). The memory 44 may store a native operating system, one or more native applications, emulation systems, or emulated applications for any of a variety of operating systems and/or emulated hardware platforms, emulated operating systems, etc. In some embodiments, a separate storage device (STOR DEV) may be coupled to the data bus 46 or as a network-connected device (or devices) via the I/O interfaces 40 and the Internet 20. The storage device may be embodied as persistent memory (e.g., optical, magnetic, and/or semiconductor memory and associated drives) to store user data (e.g., based on questionnaires, recorded data communicated from the wearable device 12, and/or via data entered in web pages accessed at the electronics devices 14, 16).

In the embodiment depicted in FIG. 3, the memory 44 comprises an operating system 50 (OS), and application software 52 (APP SW), which in some embodiments may comprise all or a portion of the functionality of the application software residing in the wearable device 12, and interface software (e.g., including one or more APIs) for enabling access by one or more devices over the Internet and/or other networks.

Execution of the application software 52 may be implemented by the processor 38 under the management and/or control of the operating system 50. The processor 38 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device 36.

The I/O interfaces 40 comprise hardware and/or software to provide one or more interfaces to the Internet 20, as well as to other devices such as the display screen 42 and user interfaces. In other words, the I/O interfaces 40 may comprise any number of interfaces for the input and output of signals (e.g., analog or digital data) for conveyance of information (e.g., data) over various networks and according to various protocols and/or standards. The user interfaces may include a keyboard, mouse, microphone, immersive head set, etc., which enable input and/or output by an administrator or other user.

When certain embodiments of the computing device 36 are implemented at least in part with software (including firmware), as depicted in FIG. 3, it should be noted that the software (e.g., including the application software 52) can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device 36 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

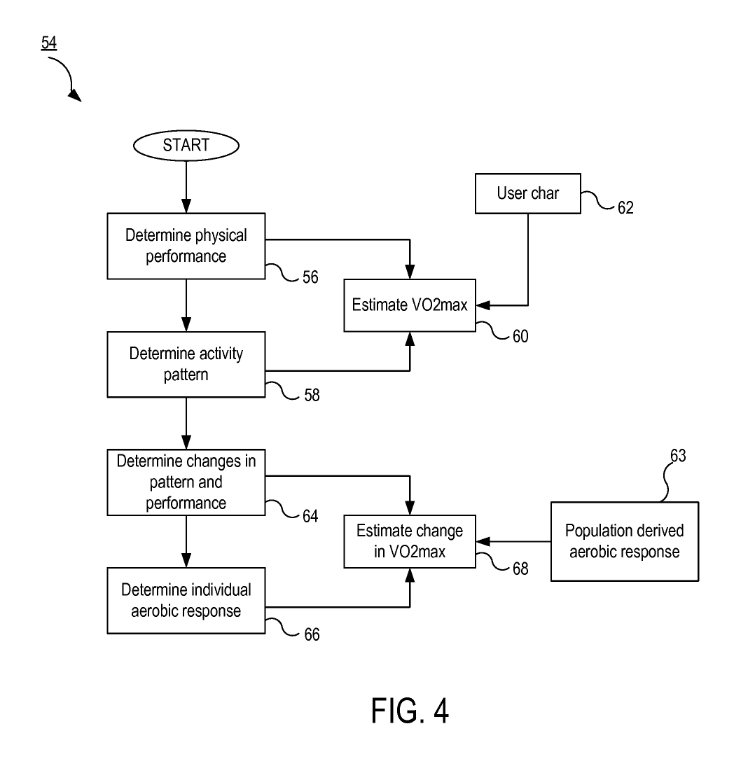

Attention is now directed to FIGS. 4-6, which illustrate various methods utilized by an embodiment of a fitness tracking system. In one embodiment, the methods depicted in the FIGS. 4-6 (and subsequently) may be performed by the processing circuit 28 (FIG. 2) executing the application software, though some embodiments may use other processing circuits and/or processor(s) of other and/or additional devices. Any process descriptions or blocks in the flow diagram shown in FIGS. 4-6 should be understood as representing modules, segments, or portions of code which include one or more executable instructions for implementing specific logical functions or steps in the process, and alternate implementations are included within the scope of an embodiment of the present invention in which functions may be executed substantially concurrently and/or in a different order, and/or additional logical functions or steps may be added, depending on the functionality involved, as would be understood by those reasonably skilled in the art of the present invention. FIG. 4 is a flow diagram that illustrates an example method 54 to determine cardiorespiratory fitness and changes in fitness from measurements of physical performance and activity patterns over time in accordance with an embodiment of the invention. The method 54 comprises, after activation (Start), determining physical performance (56) and activity patterns (58) and estimating VO₂max (60). The estimation of VO₂max may further be based on user characteristics (e.g., user-specific information) (62). The method 54 further comprises determining changes in activity patterns and performance (64) and determining individual aerobic response (66) and estimating changes in VO₂max (68). The estimate of changes may be based on population derived aerobic response (63). In general, the method 54 combines features of the user that describe (i) physical performance and (ii) activity patterns to estimate cardiorespiratory fitness and changes in fitness over time. In this way, the prediction of VO₂max is less dependent from static measurements of subjects' characteristics, which are useful to explain between individuals differences (like body weight, age or muscle mass) in VO₂max but are insensitive to temporal changes in fitness. A prediction algorithm (e.g., as described below in association with αVO₂max) used in certain embodiments of the fitness tracking system is designed to be adaptive over time, enabling a different operation according to the information gathered over time from the user. For example, VO₂max estimates are influenced by data obtained at different time periods: while physical performance is instantaneously assessed, behavioural characteristics or activity patterns are determined over long time periods. Additionally, the individual fitness response to aerobic exercise is incorporated in later stages in the prediction algorithm. A physiological response of cardiorespiratory fitness to exercise is biologically determined and can vary from a user to another, thus, personalized parameters are used to better describe long-term trends in VO₂max in response to changes in behavior or patterns and aerobic exercise engagement.

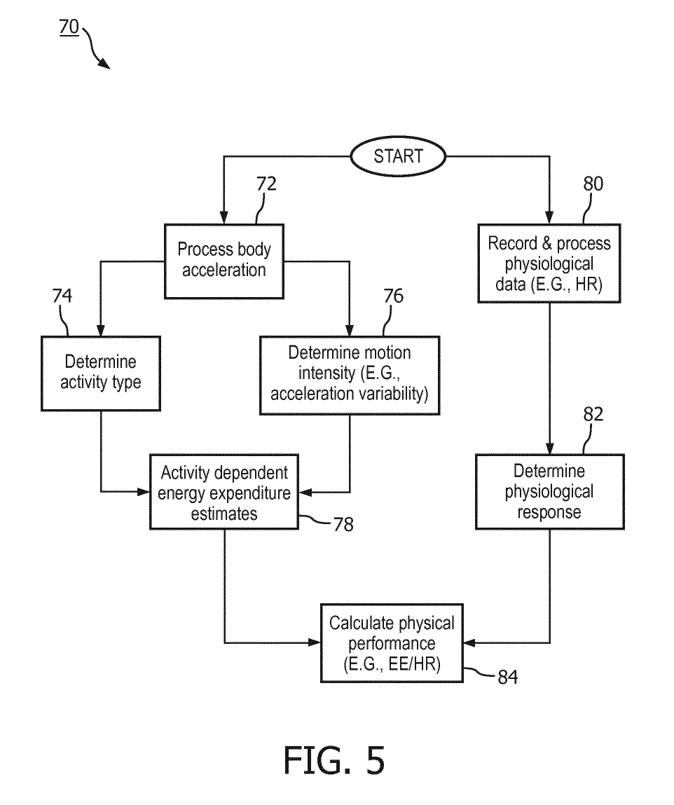

Describing FIG. 4 in further detail, and as to determining physical performance (56), one method 70, depicted in FIG. 5, is initialized to determine the user physical performance. As depicted in FIG. 5, two processing branches are shown, wherein the branch to the left in FIG. 5 comprises processing body acceleration (72), determining activity type (74) and motion intensity (e.g., acceleration variability) (76), and determining activity-dependent energy expenditure estimates based on the determinations in 74, 76 (78). In the other processing branch of FIG. 5, the method 70 records and processes physiological data (e.g., heart rate) (80), determines a physiological response (82), and calculates physical performance (e.g., total energy expenditure per heart rate (EE/HR)) (84) based on the processing in 78 and 82. Note that in some embodiments, motion intensity may be estimated from other measurements (e.g., other than movement data). For instance, in some embodiments, motion intensity may be determined from measurements from an atmospheric pressure sensor, sweat sensor, etc. Physical performance is defined as the ratio between the (a) metabolic demand and (b) the physiological response during physical activity. The metabolic demand (a) is determined by the mechanical work during physical activity, which can be estimated from body movement data, depending on the type of activity carried out by the user. For example, activity-type selective algorithms can be used to estimate energy expenditure (EE) during a specific activity (e.g. walking, cycling, etc.) from summary measures of body acceleration (e.g., activity counts, movement speed, motion cadence, periodicity, etc.). The physiological response (b) instead is defined as the level of physiological variables recorded during physical activity. This parameter can be defined as the heart rate (HR) during an activity or as a combination of multiple physiological parameters such as HR, respiration rate, skin temperature, and galvanic skin response. Physical performance (e.g., EE/HR) is designed to be equally informative irrespective of the activity type carried out by the user. In this way every time a wearable sensor detects an activity the system is capable of determining physical performance of the user associated to such activity.

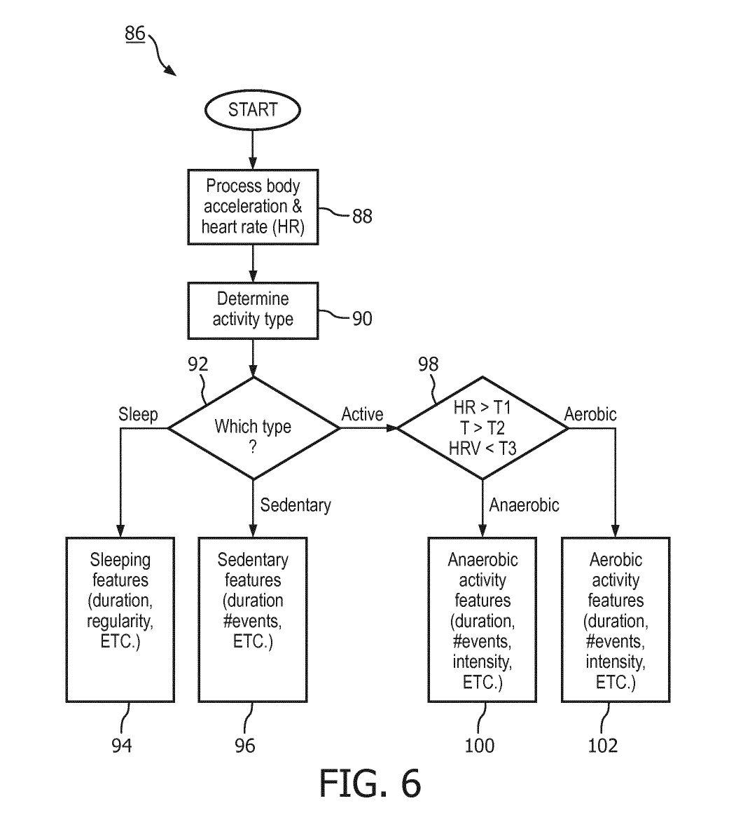

Referring now to FIG. 6, shown is a flow diagram that illustrates an example method 86 that is used to determine activity and aerobic behaviour or patterns (e.g., the activity patterns (58) of FIG. 4) from measurements of body movement and heart rate in accordance with an embodiment of the invention. In effect, the method 86 provides an activity classification that addresses multiple types of activities and their patterns in terms of duration, repetition, regularity, etc. Activity patterns can be determined by monitoring physical activity and physiological data, such as HR, over time. In the example method 86, the method 86 processes body acceleration and heart rate (HR) (88), and determines activity type (90). For instance, in (92), a determination is made that the activity is either sleep (e.g., based on sleeping features corresponding to duration, regularity, etc.) (94), sedentary (e.g., based on sedentary features of duration, number of events, etc.) (96), or active. If active, a further determination is made (98) based on heart rate, period, and heart rate variability (HRV) according to defined thresholds, and the determination is one of anaerobic (e.g., based on anaerobic features of duration, number of events, intensity, etc.) (100) or aerobic (e.g., based on aerobic features of duration, number of events, intensity, etc.) (102). Note that, from FIG. 6, T indicates activity duration; HRV indicates variability in HR over time; t₁, t₂, t₃ indicate thresholds to determine whether an activity is of an aerobic or anaerobic type. In one embodiment, aerobic activities are characterized by an HR in excess of a certain threshold (e.g. 50% HRmax), have a long duration (e.g. >30 seconds), and HRV is typically low. Activity patterns can be defined as daily duration of activity and sedentary occupations, sleeping and aerobic exercise. Activity, sedentary occupations and sleeping can be determined by processing with a classification algorithm the accelerometer signal. An algorithm processing activity duration and HR data over time automatically determines aerobic activities. For instance, when a certain activity has been carried out for a sufficient amount of time (e.g., duration >30 sec) and the HR reaches a sufficiently high value (e.g. HR >50% max HR or HR >150% resting HR) and HR variability is relatively low, the activity can be considered of an aerobic nature. Aerobic activities are particularly interesting in defining the activity patterns since these are able to maintain and modify the fitness level of the user. Activity patterns are typically assessed on a long time period such as a seven (7) consecutive days, given the repetitive and weekly nature of users' activity routine and schedule, though other periods may be used in some embodiments.

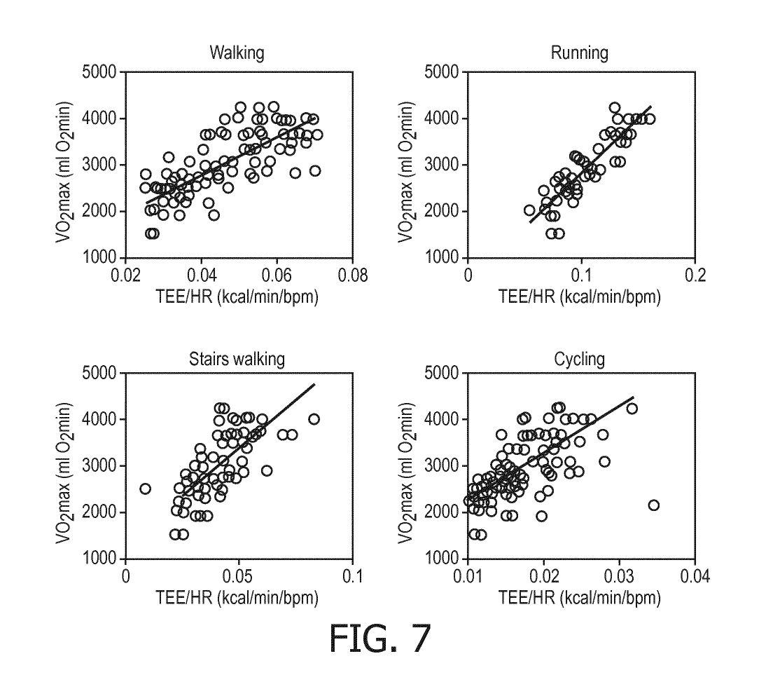

As to the estimate of VO₂max (60, FIG. 4), a context-dependent assessment of the initial VO₂max is implemented. The initial fitness level of a user can be determined by estimating VO₂max from measurements of physical performance, activity patterns, and the characteristics of a subject. Context-dependent (or activity-type dependent) regression equations can be used to estimate VO₂max from the physical performance parameter (e.g. EE/HR). Given that physical performance is determined for multiple types of daily activities, a VO₂max prediction equation can be designed to account for which activity type (a={act_1, act_2, . . . , act_A}) was carried out. Note that “a” is an index in the equation below (VO₂max equation) and indicates the activity type such as light, moderate, vigorous intensity activities or alternatively walking, running, biking, rowing, etc. Reliability weights (relₐ) can be designed to give less relevance to the predictions obtained during certain activities for which the derived physical performance is less descriptive of VO₂max, as indicated by the stronger relationships that exist between physical performance (e.g., TEE/HR) and fitness (VO₂max) depicted in FIG. 7 and in Table 1. Table 1 below illustrates VO2max prediction algorithms (e.g., correlation and error statistics) based on the TEE/HR feature for different clustering systems used to represents the free-living activity patterns. The correlation and error statistics can be used to determine the reliability score of each cluster to generate a daily VO₂max estimate.

L1SO CV

| | | | |
|---|---|---|---|
| | | Bias | RMSE |
| Cluster type | R² | (ml O₂/min) | (ml O₂/min) |

Activity type clustering

| | | | |
|---|---|---|---|
| Others | 0.67 | −0.45 | 401.17 |
| Sedentary | 0.57 | −4.87 | 459.93 |
| Ambulatory | 0.73 | 0.61 | 360.09 |

Motion intensity clustering

| | | | |
|---|---|---|---|
| Cnts <25th percentile | 0.61 | −3.50 | 439.85 |
| 25th perc ≤ Cnts < 50th | 0.49 | −8.87 | 512.06 |
| perc | | | |
| 50th perc ≤ Cnts < 75th | 0.68 | −2.18 | 395.93 |
| perc | | | |
| Cnts ≥75th perc | 0.55 | 3.67 | 474.19 |

Activity level clustering

| | | | |
|---|---|---|---|
| PAL <1.5 | 0.58 | −4.90 | 459.24 |
| 1.5 ≤ PAL < 3 | 0.59 | −0.87 | 449.56 |
| 3 ≤ PAL < 6 | 0.72 | −0.39 | 367.71 |
| PAL ≥6 | 0.82 | −1.14 | 300.38 |

k-NN clustering

| | | | |
|---|---|---|---|
| Cluster 1 | 0.73 | −2.01 | 367.64 |
| Cluster 2 | 0.45 | −6.36 | 515.28 |
| Cluster 3 | 0.70 | −0.30 | 381.32 |
| Cluster 4 | 0.83 | −1.45 | 302.47 |
| Cluster 5 | 0.77 | −0.89 | 333.48 |

Note that R² = correlation between measured and predicted VO2max, L1SO = leave-one-subject-out cross-validation error statistics, RMSE = root mean squared error, Cnts = activity counts per minute, and PAL = physical activity level.

The fitness measure derived for each activity type (act_1, act_2, etc.) is weighted according to a reliability score (relₐ) to generate a daily estimate of VO₂max. Act_A indicates the total category with which the activity types are discretized. FIG. 7 illustrates a relationship between EE/HR and VO₂max, which may be characterized as fa(EE/HR, age, weight, gender, height). The reliability scores can be defined as the correlation coefficients between EE/HR and VO₂max for each a-activity type as visible from the data shown in FIG. 7. In general, the different relationships that exist between physical performance and fitness indicate that different reliability weights can be used to generate optimal fitness prediction according to the activity type used to determine physical performance. Similarly, coefficients of VO₂max prediction equations can be made pattern or behaviour-dependent to allow estimates of fitness dependent on the amount of aerobic activity or sedentary time of a user. In this way, more tailored prediction models (e.g., to estimate changes in VO₂max over time from information on aerobic activity intensity, initial VO₂max(t₀), and population-derived parameters to run the equation associated with VO₂max (tₙ), as described further below) for groups of users can be defined and higher accuracy is achieved for predicting VO₂max.

$${V O}_{2} max = \frac{{\sum }_{a = 1}^{A} {rel}_{a} \times {f}_{a} ( \frac{EE}{HR} , age , weight , gender , height )}{{\sum }_{a = 1}^{A} {rel}_{a}}$$

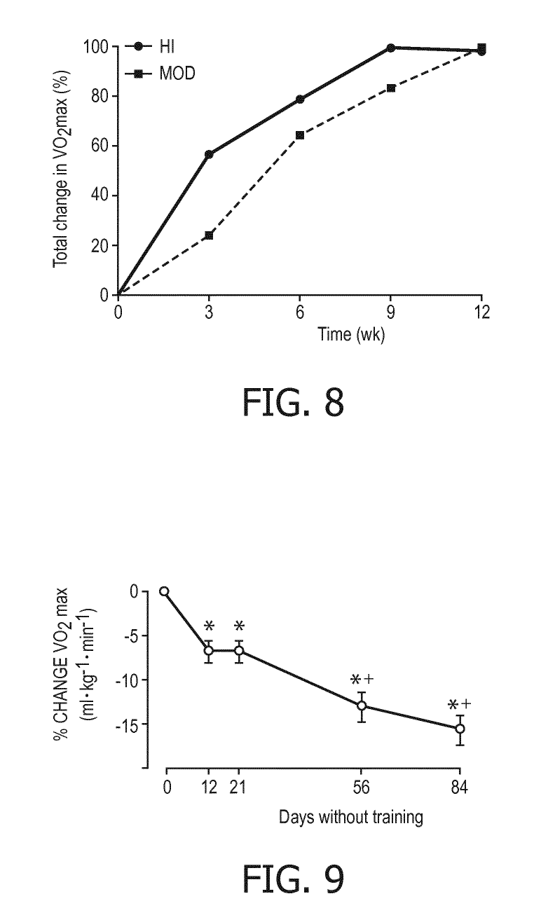

As to the estimation of changes in VO₂max (68, FIG. 4), changes in VO₂max are predicted by considering (i) the initial VO₂max, (ii) the change in VO₂max predicted according to physical performance and (iii) the fitness change (temporal trends in VO₂max) determined according to changes in activity patterns. Indeed, increases in aerobic activity can stimulate predictable changes in VO₂max, such as shown in FIG. 8. As shown, moderate intensity aerobic exercise induces a smoother change in fitness over time as compared to high intensity aerobic exercise. Similarly, a diminution in aerobic time or aerobic activity intensity may cause reduction in VO₂max, as shown in FIG. 9. The magnitude of the fitness response to changes in aerobic activity patterns is subjectively determined given the heterogeneity of VO₂max training response, as described previously. Initially, the aerobic exercise response of a user is considered equal to the population average, and only in later stages derived from the recorded data as explained below.

Estimates of VO₂max derived from physical performance are expected to fluctuate substantially over time due to day-to-day differences in physiological conditions, which are influenced by daily factors like sleep deprivation and duration, stress, over- or under-eating, hormonal cycles, and post-exercise recovery. To mitigate these non-fitness related fluctuations and improve reliability of VO₂max estimates over time, information on activity patterns and especially aerobic exercise features are used by an embodiment of a fitness tracking system to predict trajectories in fitness trends around which to expect variations in VO₂max over time. Activity patterns are assessed on a long time period according to the users' routine. Typically, patterns are determined on a weekly or bi-weekly basis to capture characteristics influenced by week and weekend days.

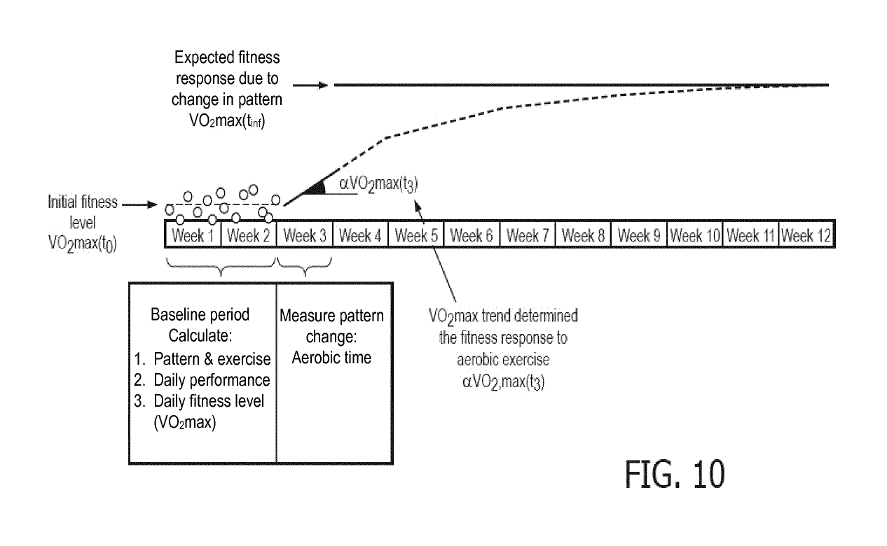

Referring now to FIG. 10, in a baseline period, the initial behaviour or pattern of the user is determined as described in association with FIG. 6. Aggregated results over a 1- or 2-week time period are used to establish the typical activity patterns (e.g., calculations are performed for behaviour or activity patterns and exercise, daily performance, daily fitness level (e.g., VO₂max)). In effect, the baseline period enables a determination of a reference starting level for periodic (e.g., weekly) VO₂max from a sequence of daily VO₂max estimates. Special emphasis is given in this description to aerobic activity intensity and duration, however, other aspect of behaviour or patterns may be considered as relevant to predict changes in fitness. For instance, anaerobic exercise may be disregarded as to considerations of meaningful activity to increase VO₂max. However, other aspects of behaviour or patterns, such as sedentary time, number of steps, etc. can replace the duration and intensity of aerobic activity to predict changes in VO₂max over time from the baseline value. An initial fitness level (VO₂max(t₀)) is determined by assessing day-to-day VO₂max estimates according to physical performance and initial patterns or behaviour (as previously described). Measurements of behavioural or pattern change are derived in the following period (e.g., in the third week). Note that the duration of the baseline and change periods may be different than those described in the example above in some embodiments. In case a user begins a training program and his/her aerobic activity increases, an embodiment of the fitness tracking system determines the expected change over time in fitness according to the magnitude of the behavioural or pattern change, as depicted in FIG. 10.

VO₂max over time can be estimated by considering: amount of aerobic activity, intensity of aerobic activity, time from the start of training, initial VO₂max level, and subject's characteristics. An example of such a model is the following:

VO₂max(tₙ)=VO₂max(t₀)+(Va₂max(t_{inf})−VO₂max(t₀))×(1−e^{-t}^{ₙ}^{/T})

where tₙ is the current time (in days) from the start of the training program, t_{inf} is the asymptotic estimated change in fitness triggered by the behavioural or pattern change; and T is the time constant that determines the amount of time (in days) needed for the user to reach 63.2% of the expected final VO₂max (e.g. VO₂max(t_{inf})). The immediately above equation models the expected VO₂max at time, tₙ. Through simple math manipulation, an expression for the change in VO₂max may be defined as VO₂max(tₙ)−VO₂max(t₀). The relationship between the behavioural or pattern change and VO₂max(t_{inf}) is initially derived form group statistics of published fitness response to training (e.g., FIGS. 8-9). The personal fitness response to exercise and VO₂max(t_{inf}) can be determined from user data in the first period following behavioural or pattern change. For instance, after an initial phase the measurements from the wearable sensors can be used to generate person-specific trends that describe the individual response to aerobic training (or de-training). Similarly, T is initially determined by using population-derived averages of fitness response to training. For example, in case of vigorous aerobic training, T is 25 days (>3 weeks), while for moderate intensity aerobic training T is 42 days (6 weeks). The initial trend in the variation of VO₂max over time can be determined as the differential over time in the equation above.

$$α {VO}_{2} max ( {t}_{3} ) = \frac{d {VO}_{2} max ( t )}{dt} evaluated at {t}_{3}$$

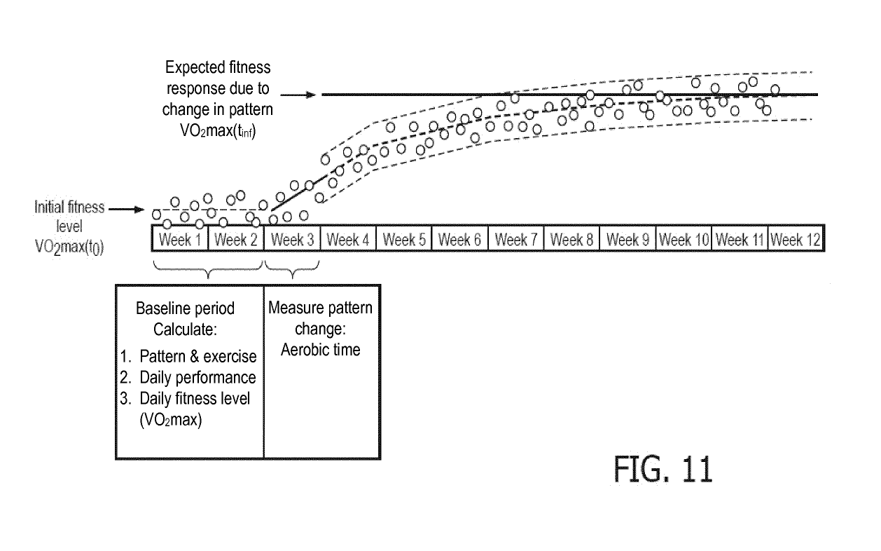

Once the parameters of the model (VO₂max(t_{inf}), and T) describing changes in fitness over time have been determined, the trajectory of reliable fitness change can be established, as represented by the dashed line in FIG. 10. This process represents a personalization process for the T parameter used to predict the temporal variation in VO₂max. In this way a better estimation of the changes in VO₂max can be obtained according to the user-specific response to changes in aerobic activity patterns. Daily estimates of fitness can be modified to remain within a certain reliability boundary around the expected fitness change, such as shown by the boundary lines above and below the initial trajectory line in FIG. 11. The reliability boundaries may be defined as a percentage of the expected VO₂max change trajectory (e.g., 90%-110% of the expected value to determine lower and upper boundaries). In this way daily estimates of fitness are more accurate as dependent on the changes in activity patterns of the user as well as the daily physical performance. In general, different approaches may be used to deploy insights into temporal trends in VO₂max due to activity patterns to adjust daily estimates. In one example approach, a baseline correction of the estimates may be applied to match an expected weekly temporal trend. In another example approach, boundary thresholds (e.g., upper and lower) may be generated, wherein daily estimates may be clamped to these boundary thresholds to allow daily estimates to still represent an expected cardio fitness change trend.

Changes in the training regime, and reduction in aerobic time, determine variations in the temporal trends in VO₂max which can still be modelled by the method presented above.

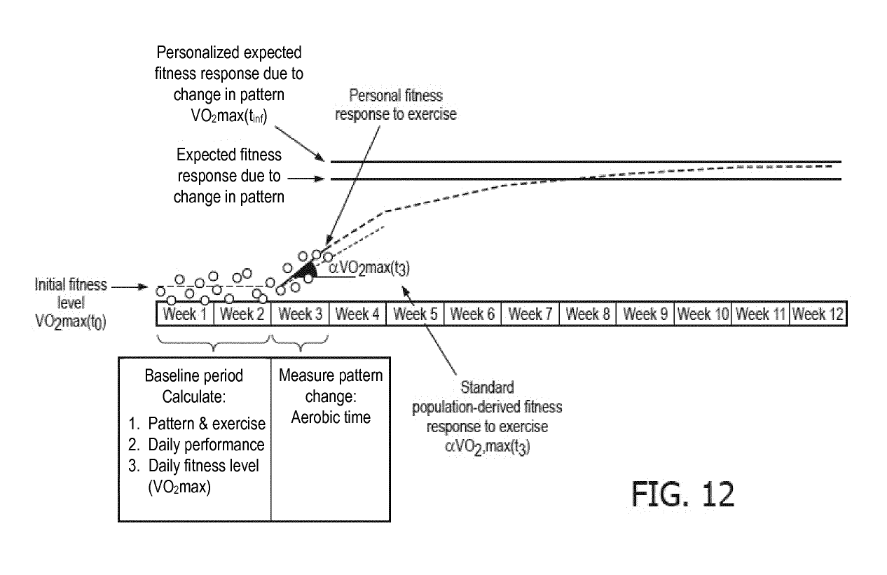

With reference to FIG. 12, shown is an illustration of prediction of an aerobic exercise response. The individual fitness response to aerobic training may be determined from user data in the first period of measurement as soon as a pattern change is recorded. As depicted in FIG. 12, it is observable that the expected training response αVO₂max differs from the actual one, as obtained by interpolation of data by a fitting line. In particular, αVO₂max indicates the first derivative over time of the equation describing changes in fitness over time (VO₂max(tₙ)), described above. αVO₂max may be used with simple arithmetic calculations to determine T, which is the time a user needs to reach 63% of the expected final VO₂max at regime (t_{inf}). Initially, this time T is obtained from population averages, but after the first period (e.g., one week), the daily estimates of VO₂max (as provided by raw data from the VO₂max equation described previously, and this uncorrected for the expected fitness changes according to the change in aerobic behavior) can be used to determine the slope of the increase in VO₂max, which corresponds to the personalized αVO₂max. This personalization process enables the fitness tracking system to update the coefficients of the model, predicting changes in fitness over time. In this way, the personalized fitness response due to pattern change (VO₂max(t_{inf})) can be calculated from αVO₂max given a certain T determined by the change in aerobic activity and pattern.

In one embodiment, a claim to a method is disclosed, and comprises receiving data obtained from wearable sensors coupled to a subject; and based on the data and subject characteristics, estimating a cardiorespiratory fitness measure for the subject by determining physical performance for free-living activities associated with free-living activities categories over a defined period of time, wherein the physical performance determination for each of the activities is indicated by a ratio between a measure of mechanical work corresponding to the activity and the activity category and a measure of a physiological response associated with the mechanical work.

In one embodiment, a claim to the method described above is disclosed, and further comprises estimating a change in the determined cardiorespiratory fitness measure for the subject based on changes in the physical performance and changes in an activity pattern and the estimated cardiorespiratory fitness measure.

In one embodiment, a claim depending on any one of the preceding method claims is disclosed, wherein the data comprises movement data and physiological data corresponding to the subject, wherein determining physical performance comprises: determining activity type and motion intensity; determining heart rate based on the physiological data; and determining activity-dependent energy expenditure estimates based on the activity type and the motion intensity, wherein the physical performance comprises a ratio of the energy expenditure estimates and the heart rate.

In one embodiment, a claim depending on any one of the preceding method claims is disclosed, wherein the movement data comprises acceleration data corresponding to the subject.

In one embodiment, a claim depending on any one of the preceding method claims is disclosed, further comprising determining an activity pattern from the free-living activities by categorizing the activity as sleep, sedentary, or active, the categorization based on the movement data and the physiological data.

In one embodiment, a claim depending on any one of the preceding method claims is disclosed, further comprising determining whether the active categorization corresponds to anaerobic or aerobic activity based on a duration of the activity and plural thresholds for the physiological data.

In one embodiment, a claim depending on any one of the preceding method claims is disclosed, wherein estimating the cardiorespiratory fitness measure comprises: estimating the cardiorespiratory fitness measure over the defined period of time for multiple types of activities for which the data is received and weighting each of the multiple types of activities according to a reliability score, the reliability score comprising correlation coefficients between the physical performance and the cardiorespiratory fitness measure.

In one embodiment, a claim depending on any one of the preceding method claims is disclosed, wherein the defined period of time consists of a day of activities.

In one embodiment, a claim depending on any one of the preceding method claims is disclosed, further comprising estimating the cardiorespiratory fitness measure for plural successive defined periods of time for a duration of a baseline period.

In one embodiment, a claim depending on any one of the preceding method claims is disclosed, further comprising estimating a change in the determined cardiorespiratory fitness measure for the subject based on changes in the physical performance and changes in an activity pattern and the estimated cardiorespiratory fitness measure after the baseline period.

In one embodiment, a claim depending on any one of the preceding method claims is disclosed, wherein a relationship between the pattern change and the cardiorespiratory fitness measure is derived from population group statistics and corrected using data from the wearable sensors.

In one embodiment, a claim depending on any one of the preceding method claims is disclosed, further comprising measuring an aerobic exercise response related to a measured pattern change.

In one embodiment, a claim to an apparatus is disclosed, the apparatus comprising: wearable sensors coupled to a subject; and a processing circuit coupled to the wearable sensors, the processing circuit configured to: receive data obtained from wearable sensors; and based on the data and subject characteristics, estimating a cardiorespiratory fitness measure for the subject by determining physical performance for free-living activities associated with free-living activities categories over a defined period of time, wherein the physical performance determination for each of the activities is indicated by a ratio between a measure of mechanical work corresponding to the activity and the activity category and a measure of a physiological response associated with the mechanical work.

In one embodiment, a claim depending the preceding apparatus claim is disclosed, wherein the processing circuit is further configured to: estimate a change in the determined cardiorespiratory fitness measure for the subject based on changes in the physical performance and changes in an activity pattern and the estimated cardiorespiratory fitness measure.

In one embodiment, a claim depending on any one of the preceding apparatus claims is disclosed, wherein the processing circuit is further configured to: determine activity type and motion intensity; determine heart rate based on the physiological data; and determine activity-dependent energy expenditure estimates based on the activity type and the motion intensity, wherein the physical performance comprises a ratio of the energy expenditure estimates and the heart rate.

In one embodiment, a claim depending on any one of the preceding apparatus claims is disclosed, wherein the processing circuit is further configured to: determine an activity pattern by categorizing each of the free-living activities as sleep, sedentary, or active, the categorization based on movement data and physiological data; and determine whether the active categorization corresponds to anaerobic or aerobic activity based on a duration of the activity and plural thresholds for the physiological data.

In one embodiment, a claim depending on any one of the preceding apparatus claims is disclosed, wherein the processing circuit is further configured to: estimate the cardiorespiratory fitness measure over the defined period of time for multiple types of activities for which the data is received and weighting each of the multiple types of activities according to a reliability score, the reliability score comprising correlation coefficients between the physical performance and the cardiorespiratory fitness measure; and repeat the estimate for plural successive defined periods of time for a duration of a baseline period.

In one embodiment, a claim depending on any one of the preceding apparatus claims is disclosed, wherein the processing circuit is further configured to: estimate a change in the determined cardiorespiratory fitness measure for the subject based on changes in the physical performance and changes in an activity pattern and the estimated cardiorespiratory fitness measure after the baseline period, wherein a relationship between the pattern change and the cardiorespiratory fitness measure is derived from population group statistics and corrected using data from the wearable sensors; and measure an aerobic exercise response to determine the pattern change.

In one embodiment, a claim to a non-transitory computer readable medium is disclosed, wherein the non-transitory computer readable medium is encoded with instructions executable by a processor or processors that causes the processor or processors to: receive data obtained from wearable sensors coupled to a subject; and based on the data and subject characteristics, estimate a cardiorespiratory fitness measure for the subject by determining physical performance for free-living activities associated with free-living activities categories over a defined period of time, wherein the physical performance determination for each of the activities is indicated by a ratio between a measure of mechanical work corresponding to the activity and the activity category and a measure of a physiological response associated with the mechanical work.

In one embodiment, a claim depending on the preceding non-transitory computer readable medium is disclosed, wherein the encoded instructions are executable by the processor or processors to cause the processor or processors further to: estimate a change in the determined cardiorespiratory fitness measure for the subject based on changes in the physical performance and changes in the activity pattern and the estimated cardiorespiratory fitness measure.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

1. A method, comprising:
receiving data obtained from wearable sensors coupled to a subject; and
based on the data and subject characteristics, estimating a cardiorespiratory fitness measure for the subject by determining physical performance for free-living activities associated with free-living activities categories over a defined period of time, wherein the physical performance determination for each of the activities is indicated by a ratio between a measure of mechanical work corresponding to the activity and the activity category and a measure of a physiological response associated with the mechanical work.

2. The method of claim 1, further comprising estimating a change in the determined cardiorespiratory fitness measure for the subject based on changes in the physical performance and changes in an activity pattern and the estimated cardiorespiratory fitness measure.

3. The method of claim 1, wherein the data comprises movement data and physiological data corresponding to the subject, wherein determining physical performance comprises:
determining activity type and motion intensity;
determining heart rate based on the physiological data; and
determining activity-dependent energy expenditure estimates based on the activity type and the motion intensity, wherein the physical performance comprises a ratio of the energy expenditure estimates and the heart rate.

4. The method of claim 3, wherein the movement data comprises acceleration data corresponding to the subject.

5. The method of claim 3, further comprising determining an activity pattern from the free-living activities by categorizing the activity as sleep, sedentary, or active, the categorization based on the movement data and the physiological data.

6. The method of claim 5, further comprising determining whether the active categorization corresponds to anaerobic or aerobic activity based on a duration of the activity and plural thresholds for the physiological data.

7. The method of claim 1, wherein estimating the cardiorespiratory fitness measure comprises:
estimating the cardiorespiratory fitness measure over the defined period of time for multiple types of activities for which the data is received and weighting each of the multiple types of activities according to a reliability score, the reliability score comprising correlation coefficients between the physical performance and the cardiorespiratory fitness measure.

8. The method of claim 7, wherein the defined period of time consists of a day of activities.

9. The method of claim 7, further comprising estimating the cardiorespiratory fitness measure for plural successive defined periods of time for a duration of a baseline period.

10. The method of claim 9, further comprising estimating a change in the determined cardiorespiratory fitness measure for the subject based on changes in the physical performance and changes in an activity pattern and the estimated cardiorespiratory fitness measure after the baseline period.

11. The method of claim 10, wherein a relationship between the pattern change and the cardiorespiratory fitness measure is derived from population group statistics and corrected using data from the wearable sensors.

12. The method of claim 11, further comprising measuring an aerobic exercise response related to a measured pattern change-.

13. An apparatus, comprising:
wearable sensors coupled to a subject; and
a processing circuit coupled to the wearable sensors, the processing circuit configured to:
receive data obtained from the wearable sensors; and
based on the data and subject characteristics, estimate a cardiorespiratory fitness measure for the subject by determining physical performance for free-living activities associated with free-living activities categories over a defined period of time, wherein the physical performance determination for each of the activities is indicated by a ratio between a measure of mechanical work corresponding to the activity and the activity category and a measure of a physiological response associated with the mechanical work.

14. (canceled)

15. (canceled)

16. (canceled)

17. (canceled)

18. (canceled)

19. A non-transitory computer readable medium encoded with instructions executable by a processor or processors that causes the processor or processors to:
receive data obtained from the wearable sensors; and
based on the data and subject characteristics, estimate a cardiorespiratory fitness measure for the subject by determining physical performance for free-living activities associated with free-living activities categories over a defined period of time, wherein the physical performance determination for each of the activities is indicated by a ratio between a measure of mechanical work corresponding to the activity and the activity category and a measure of a physiological response associated with the mechanical work.

20. (canceled)
